(19) United States
(12) Patent Application Publication
(10) Pub. No.: US 2009/0131470 A1
(43) Pub. Date: May 21, 2009

(54) PYRAZOLE-SUBSTITUTED BENZIMIDAZOLE DERIVATIVES FOR USE IN THE TREATMENT OF CANCER AND AUTOIMMUNE DISORDERS

(75) Inventors: David Lee Walmsley, Winnersh (GB); Martin James Drysdale, Winnersh (GB); Christopher John Northfield, Winnersh (GB); Christophe Fromont, Winnersh (GB)

Correspondence Address: BANNER & WITCOFF, LTD., 1100 13th STREET, N.W., SUITE 1200, WASHINGTON, DC, 20005-4051, US

(73) Assignee: VERNALIS R & D LIMITED, Winnersh (GB)

(21) Appl. No.: 11/916,485

(22) PCT Filed: Jun. 6, 2006
(86) PCT No.: PCT/GB2006/002071

§ 371 Date: Jun. 6, 2008

(30) Foreign Application Priority Data
Jun. 11, 2005 (GB) 0511947.4
Apr. 18, 2006 (GB) 0607550.1

(51) Int. Cl. A61K 31/439; C07D 453/02; A61K 31/454; C07D 401/14; A61P 37/02

(52) U.S. Cl. 514/305; 546/133; 514/322; 546/199

(57) ABSTRACT

Compounds of formula (I) are inhibitors of PDK1 and CHK1 activity, and of use in the treatment of cancer and autoimmune disorders (I): wherein R₂ is a radical of formula R₇—(CH₂)ₙ₋, or a radical of formula -Alk-N(—R₅)—R₉ wherein n is 0, 1, 2 or 3 and Alk is C₁-C₆ alkylene; R₇ is (i) a heterocyclic ring of 5 or 6 ring atoms coupled via a ring carbon wherein the sole heteroatom is nitrogen, optionally substituted by C₁-C₆ alkyl or aryl C₁-C₆ alkyl, (ii) 1-aza-bicyclo[2.2.2]oct-3-yl, or (iii) 8-methyl-8-aza-bicyclo[3.2.1]oct-3-yl; R₈ and R₉ are independently selected from hydrogen or C₁-C₃ alkyl; and the remaining substituents are as defined in the claims.

This invention relates to substituted benzimidazole compounds having PDK1 and CHK1 inhibitory activity, to the use of such compounds in medicine, in relation to the treatment of disorders which are responsive to inhibition of PDK1 and CHK1 such as cancer and autoimmune disorders, and to pharmaceutical compositions containing such compounds.

### BACKGROUND TO THE INVENTION

### PDK1

For a normal cell to acquire the phenotype of a malignant tumour cell, several barriers must be overcome. One of the most important is the ability to evade programmed cell death (apoptosis). Mutations down regulating various aspects of the cell-death machinery are therefore a hallmark of cancer. The PI-3 kinase-AKT pathway transmits survival signals from growth factor receptors to downstream effectors. In a substantial number of tumour cells, this pathway is inappropriately activated by either amplification of the PI-3 kinase or Akt genes, or loss of expression of the PTEN tumour suppressor. Activation of this pathway enables cancer cells to survive under conditions where normal cells would die, enabling the continued expansion of the tumour. The 3′-phosphoinositide-dependent protein kinase-1 (PDK1) is an essential component of the PI-3 kinase-AKT pathway. In the presence of PIP3, the second messenger generated by PI-3 kinase, PDK1 phosphorylates Akt on threonine 308, a modification essential for Akt activation. PDK1 also phosphorylates the corresponding threonine residues of certain other pro-survival kinases including SGK and p70 S6 kinase (Vanhaesebroeck B & Alessi D R. Biochem J 346, 561-576 (2000)). Experiments with genetically modified mice indicate that reducing PDK1 activity to 10% of the normal level is surprisingly well tolerated (Lawlor M A et al. EMBO J. 21, 3728-3738 (2002)). Certain cancer cells, however, appear to be less able to tolerate antisense-mediated reductions in PDK1 activity (Flynn P et al. Curr Biol. 10, 1439-1442 (2000)). Moreover, both celecoxib and UCN-01, small molecules that inhibit PDK1 both in vitro and in cells, are capable of inducing apoptosis in cultured tumour cells (Arico et al. J. Biol. Chem. 277, 27613-27621 (2002); Sato et al. Oncogene 21, 1727-1738 (2002)). More recently Berlex Biosciences (Richmond USA) published PDK1 inhibitors that in-vitro arrested cell cycle at G2-M leading to apoptosis and also demonstrated in-vivo efficacy versus a mouse lung metastasis model (Journal of Biological Chemistry Mar. 16, 2005 Manuscript M501367200). Agents that inhibit the PDK1 kinase may therefore be useful for the therapy of cancer.

Further, PDK1 is implicated in T-cell function and proliferation. Alessi and co-workers explored the consequences of genetic manipulation of PDK1 (Nature Immunology 2004, 5(5), 539-545). PDK1 is a rate-limiting ‘upstream’ activator of AGC kinases. AGC family kinases are essential for T cell development. Alessi analyzed the effect of PDK1 deletion on T-cell lineage development & also assessed the consequences of reducing PDK1 levels to 10% of normal. Complete PDK1 loss blocked T cell differentiation in the thymus, whereas reduced PDK1 expression to 10% of normal, allowed T cell differentiation but blocked proliferative expansion.

Moreover, Ghosh and co-workers showed that PDK1 has an essential role in regulating the activation of PKC-theta and through signal-dependant recruiting of both PKC-theta and CARD11 to lipid rafts. PDK1-associated PKC-theta recruits the IKK complex, whereas PDK1-associated CARD11 recruits the Bc110-MALT1 complex, allowing activation of the IKK complex through Bc110-MALT1-dependant ubiquitination of the IKK complex subunit (Known as NEMO, NF-kB essential modifier). PDK1 therefore plays a critical role by nucleating the TCR-induced NF-kB activation pathway in T-cells. Agents that Inhibit PDK1 kinase may therefore be useful for the treatment of autoimmune-disorders such as organ transplant rejection, lupus, multiple sclerosis, rheumatoid arthritis & osteoarthritis.

### CHK1

Many standard cancer chemotherapeutic agents act primarily through their ability to induce DNA damage causing tumor growth inhibition. However, these agents cause cell cycle arrest by induction of checkpoints at either S-phase or G2-M boundary. The G2 arrest allows the cell time to repair the damaged DNA before entering mitosis. Chk1 and an unrelated serine/threonine kinase, Chk2, play a central role in arresting the cell cycle at the G2-M boundary (O'Connell et al EMBO J. (1997) vol 16 p545-554). Chk1/2 induce this checkpoint by phosphorylating serine 216 of the CDC25 phosphatase, inhibiting the removal of two inactivating phosphates on cyclin dependent kinases (CDKs) (Zheng et al Nature (1998) vol 395 p507-510). Another overlapping pathway mediated by p53 also elicits cycle arrest in response to DNA-damage. However, p53 is mutationally inactivated in many cancers, resulting in a partial deficiency in their ability to initiate a DNA-repair response. If Chk1 activity is also inhibited in p53-negative cancers, all ability to arrest and repair DNA in response to DNA-damage is removed resulting in mitotic catastrophe and enhancing the effect of the DNA damaging agents (Konarias et al Oncogene (2001) vol 20 p7453-7463; Bunch and Eastman Clin. Can. Res. (1996) vol 2 p791-797; Tenzer and Pruschy Curr. Med. Chem (2003) vol 3 p35-46). In contrast, normal cells would be relatively unaffected due to retention of a competent p53-mediated cell-cycle arrest pathway. A Chk1 inhibitor (UCN-01) is now in phase I clinical trials for improving the efficacy of current DNA-damage inducing chemotherapeutic regimens (Sausville et al, J. Clinical Oncology (2001) vol 19 p2319-2333).

### BRIEF DESCRIPTION OF THE INVENTION

The present invention relates to a class of substituted benzimidazole compounds useful as inhibitors of PDK1 and CHK1, for example, for the treatment of cancer. A core benzimidazole ring substituted on the heterocyclic ring with a pyrazole ring is a principle characterising feature of the compounds with which the invention is concerned.

### DETAILED DESCRIPTION OF THE INVENTION

According to the present invention, there is provided a compound of (I) or a salt, hydrate or solvate thereof

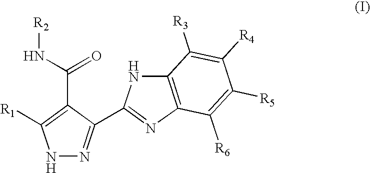

wherein
R₁ is hydrogen or C₁-C₃ alkyl;
R₂ is a radical of formula R₇—(CH₂)ₙ—, or a radical of formula -Alk-N(—R₈)—R₉ wherein n is 0, 1, 2 or 3 and Alk is C₁-C₆ alkylene;
R₃ and R₆ are independently selected from hydrogen, fluoro, or chloro;
R₄ and R₅ are independently selected from hydrogen, C₁-C₆ alkyl, halo, cyano, C₁-C₆ alkoxycarbonyl, C₁-C₆ alkoxy, trifluoromethyl, —C(═O)—NH—R₁₀, —NH—C(═O)—R₁₁, a heterocyclic ring optionally substituted by halo, or a C₃-C₆ cycloalkyl ring;
or R₄ and R₅ taken together with the carbon atoms to which they are attached form a 5- or 6-membered carbocyclic ring, or a 5- or 6-membered heterocyclic ring;
R₇ is (i) a heterocyclic ring of 5 or 6 ring atoms coupled via a ring carbon wherein the sole heteroatom is nitrogen, optionally substituted by C₁-C₆ alkyl or aryl C₁-C₆ alkyl, (ii) 1-aza-bicyclo[2.2.2]oct-3-yl, or (iii) 8-methyl-8-aza-bicyclo[3.2.1]oct-3-yl;
R₈ and R₉ are independently selected from hydrogen or C₁-C₃ alkyl;
R₁₀ and R₁₁ are independently selected from hydrogen, C₃-C₇ cycloalkyl, C₁-C₆ alkyl, C₁-C₆ alkoxy-C₁-C₆ alkyl, aryl, or aryl-C₁-C₆ alkyl wherein the C₁-C₆ alkyl part is optionally substituted by hydroxy.

The active compounds of formula (I) are inhibitors of PDK1 and CHK1 and are useful for the treatment, prevention and suppression of diseases mediated by PDK1 and CHK1. The invention is concerned with the use of these compounds to selectively inhibit PDK1 and CHK1 and, as such, in the treatment of cancer and autoimmune disorders.

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers refers to a saturated hydrocarbon chain having from a to b carbon atoms and two unsatisfied valences.

As used herein the term “cycloalkyl” refers to a saturated carbocyclic radical having from 3-8 carbon atoms and includes, for example, cyclopropyl, cyclobutyl, cyclopentyl, cyclohexyl, cycloheptyl and cyclooctyl.

As used herein the term “carbocyclic” refers to a mono- or bi-cyclic radical whose ring atoms are all carbon, and includes monocyclic aryl, cycloalkyl, and cycloalkenyl radicals, provided that no single ring present has more than 8 ring members. A “carbocyclic” group includes a mono-bridged or multiply-bridged cyclic alkyl group.

As used herein the term “aryl” refers to a mono-, bi- or tri-cyclic carbocyclic aromatic radical. Illustrative of such radicals are phenyl, biphenyl and napthyl.

As used herein the term “heteroaryl” refers to a mono-, bi- or tri-cyclic aromatic radical containing one or more heteroatoms selected from S, N and O. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the term “heterocyclic” includes “heteroaryl” as defined above, and in particular refers to a mono-, bi- or tri-cyclic non-aromatic radical containing one or more heteroatoms selected from S, N and O, to groups consisting of a monocyclic non-aromatic radical containing one or more such heteroatoms which is covalently linked to another such radical or to a monocyclic carbocyclic radical, and to a mono-, bi- or tri-cyclic non-aromatic radical containing one or more heteroatoms selected from S, N and O which is mono-bridged or multiply-bridged. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

As used herein the term “donor nitrogen atom” refers to a nitrogen atom possessing a covalently bonded hydrogen atom that has the potential to interact with a hydrogen bond acceptor, such as a carbonyl oxygen or lone pair.

As used herein the term “salt” includes base addition, acid addition and quaternary salts. Compounds of the invention which are acidic can form salts, including pharmaceutically or veterinarily acceptable salts, with bases such as alkali metal hydroxides, e.g. sodium and potassium hydroxides; alkaline earth metal hydroxides e.g. calcium, barium and magnesium hydroxides; with organic bases e.g. N-ethyl piperidine, dibenzylamine and the like. Those compounds (I) which are basic can form salts, including pharmaceutically or veterinarily acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic and p-toluene sulphonic acids and the like.

For a review on suitable salts, see Handbook of Pharmaceutical Salts: Properties, Selection, and Use by Stahl and Wermuth (Wiley-VCH, Weinheim, Germany, 2002).

The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Compounds with which the invention is concerned which may exist in one or more stereoisomeric form, because of the presence of asymmetric atoms or rotational restrictions, can exist as a number of stereoisomers with R or S stereochemistry at each chiral centre or as atropisomeres with R or S stereochemistry at each chiral axis. The invention includes all such enantiomers and diastereoisomers and mixtures thereof.

So-called ‘pro-drugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985).

Also included within the scope of the invention are metabolites of compounds of formula (I), that is, compounds formed in vivo upon administration of the drug. Some examples of metabolites include

(i) where the compound of formula (I) contains a methyl group, an hydroxymethyl derivative thereof (—CH₃—>—CH₂OH):
(ii) where the compound of formula (I) contains an alkoxy group, an hydroxy derivative thereof (—OR—>—OH);
(iii) where the compound of formula (I) contains a tertiary amino group, a secondary amino derivative thereof (—NR¹R²—>—NHR¹ or —NHR²);
(iv) where the compound of formula (I) contains a secondary amino group, a primary derivative thereof (—NHR¹—>—NH₂);
(v) where the compound of formula (I) contains a phenyl moiety, a phenol derivative thereof (-Ph->-PhOH); and
(vi) where the compound of formula (I) contains an amide group, a carboxylic acid derivative thereof (—CONH₂—>COOH).

Variable substituents present in compounds (I) will now be further defined. It is to be inferred in the further description that any disclosed substituent or substituent class may be present in any combination with any of the other disclosed substituent classes.

### The Radical R₁

R₁ is hydrogen or C₁-C₃ alkyl. Presently it is preferred that R₁ is hydrogen or methyl. Particularly preferred are those compounds wherein R₁ is methyl.

### The Radical R₂

R₂ is a radical of formula R₇—(CH₂)ₙ—, or a radical of formula -Alk-N(—R₈)—R₉. In a subclass of compounds with which the invention is concerned, n is 0 or 1 and R₇ is a heterocyclic ring of 5 or 6 ring atoms coupled via a ring carbon wherein the sole heteroatom is nitrogen, optionally substituted by C₁-C₆ alkyl or aryl-C₁-C₆ alkyl. In such cases, it is currently preferred that R₂ is piperidin-4-yl, pyrrolidin-3-ylmethyl, 1-methyl-piperidin-4-yl, or 1-aza-bicyclo[2.2.2]oct-3-yl. Particularly preferred are those compounds wherein R₂ is piperidin-4-yl, pyrrolidin-3-ylmethyl, or 1-methyl-piperidin-4-yl. In other structures, Alk may be, for example, ethyl, propyl or butyl, with R₈ and R₉ both hydrogen.

### The Radicals R₃ and R₆

R₃ and R₆ are independently selected from hydrogen, fluoro, or chloro. Currently preferred are those compounds wherein R₃ and R₆ are independently selected from hydrogen or fluoro, hydrogen being particularly preferred.

### The Radicals R₄ and R₅

R₄ and R₅ are independently selected from hydrogen, C₁-C₆ alkyl, halo, cyano, C₁-C₆ alkoxycarbonyl, C₁-C₆ alkoxy, trifluoromethyl, —C(═O)—NH—R₁₀, —NH—C(═O)—R₁₁, or a heterocyclic ring; or R₄ and R₅ taken together with the carbon atoms to which they are attached form a 5- or 6-membered carbocyclic ring, or a 5- or 6-membered heterocyclic ring. One subclass of compounds presently preferred are those wherein R₄ and R₅ are independently selected from hydrogen, methyl, fluoro, chloro, cyano, or ethoxycarbonyl. Another subclass of compounds presently preferred is that wherein R₁₀ and R₁₁ are independently selected from hydrogen, isopropyl, isobutyl, cyclopropyl, C₁-C₆ alkyl, 2-methoxyethyl, 2-phenylpropyl, or 2-phenylethyl. In other structures R₄ and R₅ may be indol-2-yl, or 2,3-dihydrobenzofuran-4-yl. It is presently preferred that R₄ and R₅ form —CH═CH—CH═CH—, —NH—CH═N—, or —NH—N═CH— when taken together to form a 5- or 6-membered ring.

According to a further aspect of the invention, there is provided for use in therapy a compound of formula (I).

According to a further aspect of the invention, there is provided the use of a compound of formula (I) in the manufacture of a medicament for the treatment of a disorder mediated by PDK1 and CHK1.

According to a further aspect of the present invention there is provided a method of treatment of a disorder mediated by PDK1 and CHK1 comprising administration to a subject in need of such treatment an effective dose of the compound of formula (I), or a pharmaceutically acceptable salt or prodrug thereof.

The disorders mediated by PDK1 and CHK1 are selected from cancer and autoimmune disorders.

The present invention is particularly directed to cancer, organ transplant rejection, lupus, multiple sclerosis, rheumatoid arthritis and osteoarthritis.

The present invention may be employed in respect of a human or animal subject, more preferably a mammal, more preferably a human subject.

As used herein, the term “treatment” as used herein includes prophylactic treatment.

The compound of formula (I) may be used in combination with one or more additional drugs useful in the treatment of the disorders mentioned above, the components being in the same formulation or in separate formulations for administration simultaneously or sequentially.

It will be understood that the specific dose level for any particular patient will depend upon a variety of factors including the activity of the specific compound employed, the age, body weight, general health, sex, diet, time of administration, route of administration, rate of excretion, drug combination and the causative mechanism and severity of the particular disease undergoing therapy. In general, a suitable dose for orally administrable formulations will usually be in the range of 0.1 to 3000 mg, once, twice or three times per day, or the equivalent daily amount administered by infusion or other routes. However, optimum dose levels and frequency of dosing will be determined by clinical trials as is conventional in the art.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

There are multiple synthetic strategies for the synthesis of the compounds (I) with which the present invention is concerned, but all rely on known chemistry, known to the synthetic organic chemist. Thus, compounds according to formula (I) can be synthesised according to procedures described in the standard literature and are well-known to the one skilled in the art. Typical literature sources are “Advanced organic chemistry”, 4^{th} Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky), review articles such as found in “Synthesis”, “Acc. Chem. Res.”, “Chem. Rev”, or primary literature sources identified by standard literature searches online or from secondary sources such as “Chemical Abstracts” or “Beilstein”. Such literature methods include those of the preparative Examples herein, and methods analogous thereto.

Suitable routes to compounds of formula (I) are shown below in schemes 1, 2 and 3.

All examples were prepared via key intermediates, either functionalised pyrazole-4-carboxylic acid esters (e.g. Ref example 3) or via corresponding pyrazole-4-carboxylic acids (e.g. Ref example 4).

Variants at R1 were introduced via choice of acylating agent in the acylation of 3-oxo-butyric acid ester, (Ref example 2) prior to cyclisation to the corresponding pyrazole with hydrazine hydrate (Ref example 3). When R1=H then the corresponding pyrazole was afforded via thermal condensation of the 3-oxo-butyric acid ester with dimethyl formamide dimethyl acetal prior to cyclisation with hydrazine.

For ease of synthesis, examples with variants R3, R4, R5 & R6 were prepared via initial carbodiimide coupling with selected amine R2 & pyrazole-4-carboxylic acid (Ref example 4). The resultant carboxamide was progressed as shown in Scheme 2, utilising a manganese dioxide oxidation to pre-form the aldehyde (Ref example 8) prior to oxidative cyclisation using sodium bisulphite as an in-situ oxidant.

A more convenient route for increased variation at R2, is shown in Scheme 3. Pyrazole-4-carboxylic acid (Ref example 4) was initially protected as the benzyl ester subsequent to similar alcohol deprotection & oxidation, prior to the oxidative cyclisation which afforded the benzimidazole pyrazole-4-carboxylic benzyl ester (Ref example 6). This could be readily converted to corresponding, late stage intermediate acid via hydrogenation (Ref example 7). Carbodiimide couplings at elevated temperatures (typically 50-70° C.) allowed rapid elaboration of R2.

Alternatively, and dependant on tolerant functionality in R3, R4, R5 & R6, the ethyl ester (Ref example 3) could be progressed in an analogous manner to that shown in scheme 3, utilising saponification prior to carbodiimide coupling.

Aromatic & heteroaromatic substitution at R4 or R5 were introduced via Suzuki coupling on 5-bromo, 2-nitroaniline prior to hydrogenation to corresponding phenylene diamine (Ref example 9) and oxidative cyclisation with appropriate aldehyde (Ref example 8).

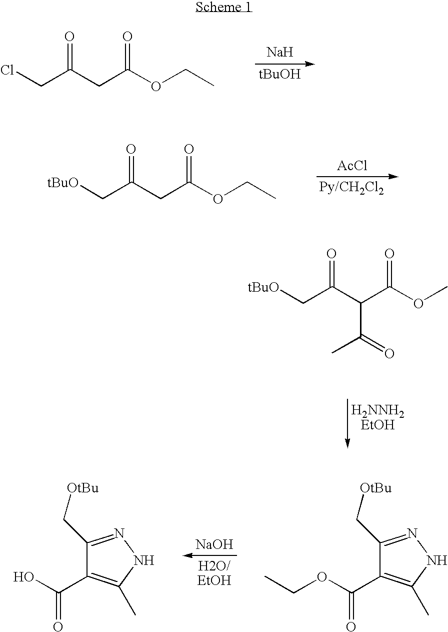

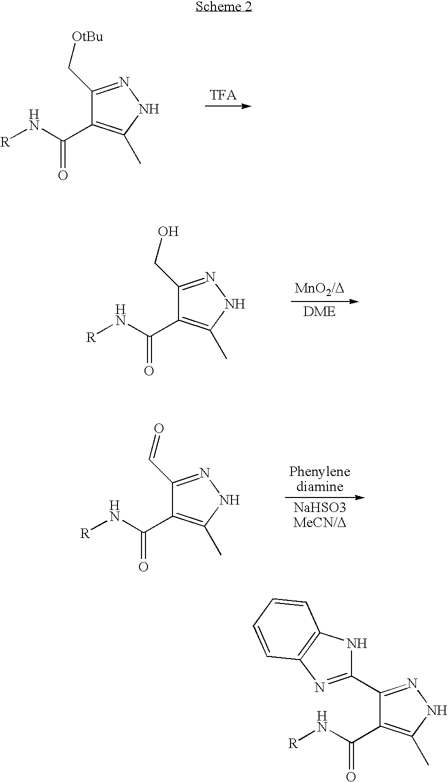

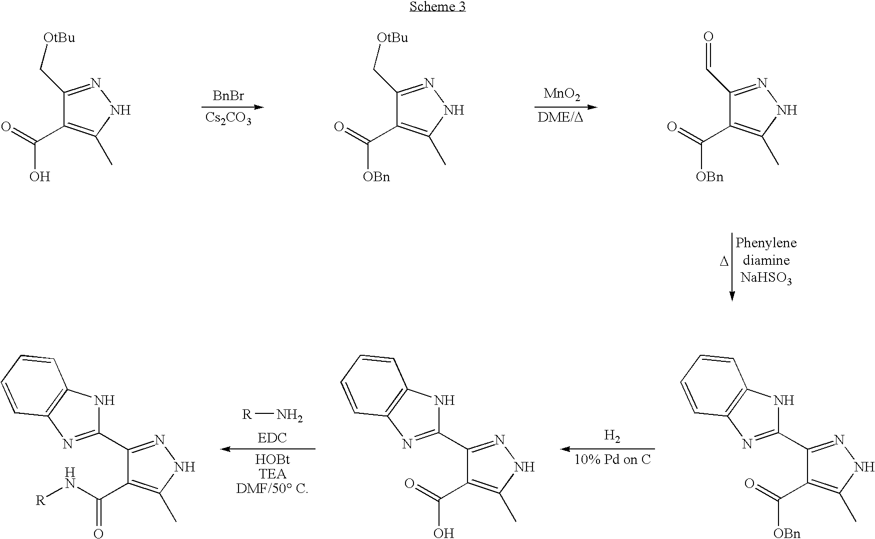

### EXAMPLES

The following examples illustrate the preparation and activities of specific compounds of the invention.

### Reference Example 1

### Synthesis of 4-tert-Butoxy-3-oxo-butyric Acid Ethyl Ester

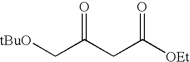

A suspension of sodium hydride (70 g 60% dispersion in mineral oil) in dimethyl formamide (400 mls) at 0° C., was treated dropwise with ethyl-4-chloroacetate (90 g) and then with tert-butyl alcohol (81 g). The mixture was maintained at 0° C. and then allowed to warm to ambient temperature over 2 hours. The mixture was poured into 2N hydrochloric acid/ice (900 mls) and then extracted three times with ethyl acetate. The combined organics were dried over magnesium sulphate and evaporated. The resultant yellow oil residue was subjected to flash column chromatography on silica eluting gradient of hexane to (1:9, v/v) ethyl acetate and hexane to give title compound (76 g) as a yellow oil. ¹H-NMR (400 MHz, CDCl3) δ H 1.20 (9H s) 1.25 (3H t), 3.54 (2H s), 4.00 (2H s), 4.19 (2H q)

### Reference Example 2

### Synthesis of 2-Acetyl-4-tert-butoxy-3-oxo-butyric Acid Ethyl Ester

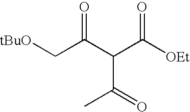

A solution of 4-tert-Butoxy-3-oxo-butyric acid ethyl ester (76 g) in dichloromethane (500 mls) was cooled in ice water bath and was treated with powdered 4 A° molecular sieves (40 g) and dry magnesium chloride (36 g). After 15 minutes the mixture was treated with pyridine (61 mls) over 5 minutes and stirring continued for 20 mins maintaining cooling. Acetyl chloride (27 mls) was added dropwise over 10 minutes with ice cooling & the whole allowed to warm to ambient temperature over 16 hours. The reaction was quenched with saturated ammonium chloride solution (300 mls) and the whole diluted with ethyl acetate (1 litre). The whole was filtered through a pad of celite and the layers partitioned. The organics were further washed with brine and dried over magnesium sulphate before being filtered and evaporated to yield 2-acetyl-4-tert-butoxy-3-oxo-butyric acid ethyl ester (88 g) as a yellow oil, which was used without further purification.

### Reference Example 3

### Synthesis of 3-tert-Butoxymethyl-5-methyl-1H-pyrazole-4-carboxylic Acid Ethyl Ester

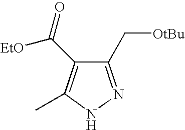

A solution of 2-acetyl-4-tert-butoxy-3-oxo-butyric acid ethyl ester (88 g) in glacial acetic acid (600 mls) was treated dropwise with hydrazine hydrate (20 mls) and the mixture stirred at ambient temperature for 16 hrs. The reaction mixture was evaporated and partitioned between ethyl acetate and saturated sodium bicarbonate solution. The organics were dried over magnesium sulphate and evaporated. The residue was eluted through a pad of silica (600 g) eluting a gradient of hexane to (1:3, v/v) ethyl acetate and hexane. Evaporation of fractions afforded title compound as a golden oil which crystallised upon standing (74 g) ¹H-NMR (400 MHz, D6 DMSO) δ H 1.19 (9H s), 1.26 (3H t), 2.34 (3H s), 4.18 (2H q), 4.54 (2H s broad).

### Reference Example 4

### Synthesis of 3-tert-Butoxymethyl-5-methyl-1H-pyrazole-4-carboxylic Acid

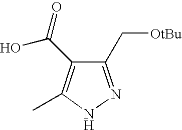

A solution of 3-tert-Butoxymethyl-5-methyl-1H-pyrazole-4-carboxylic acid ethyl ester (63 g) in ethanol (400 mls) was treated with 10% sodium hydroxide solution (aq) (800 mls) and the mixture refluxed for 16 hrs. The cooled reaction mixture was reduced in volume and further cooled in ice water bath. The pH was adjusted to <1. A cream precipitate formed which was collected by filtration and washed lightly with further water. The solid was dried in vacuum oven for 16 hrs and gave title compound (29 g) ¹H-NMR (400 MHz, DMSO) δ H 1.19 (9H s), 2.36 (3H, s), 4.58 (2H s).

### Reference Example 5

### Synthesis of 3-Hydroxymethyl-5-methyl-1H-pyrazole-4-carboxylic Acid Benzyl Ester

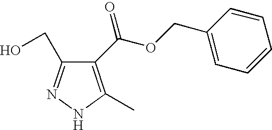

1 g (5.0 mmol) of 3-tert-Butoxymethyl-5-methyl-1H-pyrazole-4-carboxylic acid (1.0 g 5.0 mmol), was treated with cesium carbonate (0.815 g 2.5 mmol) in methanol (20 mls) was stirred under nitrogen at room temperature for 1 hr. The mixture was evaporated to dryness and the resultant salt treated with benzyl bromide (0.60 mls 5.0 mmol) in DMF (10 mls) and the whole stirred for 3 hrs. The mixture was reduced in volume and partitioned between ethyl acetate and 0.5N HCl (aq). The organic layer was further washed with 5% sodium bicarbonate (aq) and brine. The organics were dried over anhydrous magnesium sulphate, filtered and evaporated. The crude residue was treated with trifluoroacetic acid and stirred under nitrogen at room temperature for 4 hrs. The mixture was again evaporated and dried though azeotrope with toluene (3×20 mls) and the crude alcohol purified by flash column chromatography eluting ethyl acetate. Evaporation of fractions furnished desired alcohol as a colourless solid (0.61 g). LC/MS: RT 2.03 (M+H+247 & M+Na+ 269) ¹H-NMR (400 MHz CDCl₃) δ H 2.45 (3H s), 4.81 (2H s broad), 5.27 (2H s), 7.31-7.39 (5H m aromatic)

### Reference Example 6

### Synthesis of 3-(1H-Benzoimidazol-2-yl)-5-methyl-1H-pyrazole-4-carboxylic Acid Benzyl Ester

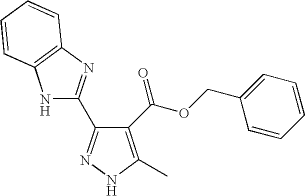

3-Hydroxymethyl-5-methyl-1H-pyrazole-4-carboxylic acid benzyl ester (0.61 g) was dissolved in DME (20 mls) and treated with manganese dioxide (2.16 g 24.8 mmol) and heated at 80° C. for 1 hr. The mixture was filtered through celite while still warm and the celite was further washed with warm methanol. The combined filtrate was evaporated and the residue treated with phenylene diamine (0.3 g 2.75 mmol) and sodium bisulphite (0.43 g 4.14 mmol) in acetonitrile (4 mls) and heated in the a microwave (Smith Synthesiser) at 160° C. for 10 mins. The reaction mixture was diluted with ethyl acetate and washed with water. The organic layer was dried with anhydrous magnesium sulphate, filtered and evaporated. The crude 3-(1H-Benzoimidazol-2-yl)-5-methyl-1H-pyrazole-4-carboxylic acid benzyl ester was purified by trituration with ethyl acetate. Collection by filtration gave title compound as a colourless solid (0.206 g). LC/MS: RT 2.18, ([M+H]⁺ 333). ¹H-NMR (400 MHz DMSO) δ H 2.50 (3H s), 5.21 (major) & 5.34 (minor) (2H s), 7.20-7.70 (9H aromatics)

### Reference Example 7

### Synthesis of 3-(1H-Benzoimidazol-2-yl)-5-methyl-1H-pyrazole-4-carboxylic Acid

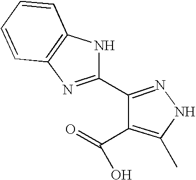

A mixture of 3-(1H-Benzoimidazol-2-yl)-5-methyl-1H-pyrazole-4-carboxylic acid benzyl ester (0.070 g 0.21 mmol) and 10% Pd/C (0.01 g catalytic) in methanol (5 mls) and dichloromethane (10 mls) was agitated under an atmosphere of hydrogen at room temperature for 1 hr. The reaction mixture was filtered through 5 micron porosity membrane filter and evaporated. The residue was triturated with methanol and filtration gave title compound as a colourless solid (0.02 g 39%), LC/MS RT: 1.74, ([M+H]⁺ 243) ¹H-NMR (400 MHz D6 DMSO) δ H 2.57 (3H s), 7.33-7.73 (4H, aromatics)

### Reference Example 8

### Synthesis of 4-[(3-Formyl-5-methyl-1H-pyrazole-4-carbonyl)-amino]-piperidine-1-carboxylic Benzyl Ester

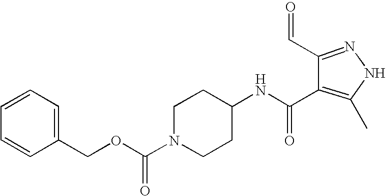

4-[(3-tert-Butoxymethyl-1H-pyrazole-4-carbonyl)-amino]-piperidine-1-carboxylic acid benzyl ester (17.39 g, 41.95 mmol) was slurried in DCM (20 mL). TFA (50 mL) was added in portions while cooling in a water bath. Three more 20 mL portions of TFA were added in 1 hour intervals and the mixture stirred at room temperature overnight. The solution was concentrated and the residue was dissolved in a mixture of THF (150 mL) and 5% Lithium hydroxide. The mixture was stirred at room temperature for 1 hour. The THF was removed under reduced pressure and the mixture was extracted twice with ethyl acetate. The organics were washed with brine and water, dried and concentrated to give a brown oil. The oil was then slurried in DME (200 mL). Manganese (II) oxide (39.6 g, 558 mmol) was added and the mixture heated to 70° C. After 1 hour the mixture was filtered hot and the residue extracted with ethyl acetate. The combined organics were concentrated and the residue partitioned between ethyl acetate and water. The organics were then washed with water and brine, dried and concentrated to give 9.90 g of a green oil which was used in subsequent reactions without further purification. LCMS (RT 2.21 minutes [M+H]⁺ 371)

### Reference Example 9

### Synthesis of 2-(3,4-Diamino-phenyl)-indole-1-carboxylic Acid Tert-Butyl Ester

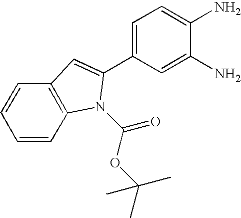

4-Bromo-2-nitroaniline (250 mg, 1.15 mmol), [1-(tert-Butoxycarbonyl)-indole-2-boronic acid (390 mg, 1.5 mmol), Sodium bicarbonate (290 mg, 6.9 mmol), and Bis(triphenylphospine)palladium (II) chloride (40 mg, 0.057 mmol) were added to DMF (10 mL). Water (1 mL) was added the mixture was degassed and heated to 90° C. under Nitrogen. After two hours the mixture was allowed to cool to room temperature whereupon water (10 mL) was added. A red precipitate was immediately formed and this was filtered, washed with water and chilled methanol to give a red solid. This solid was then taken up in methanol (20 mL) and a catalytic amount of 10% Palladium on carbon added. The mixture was hydrogenated at ambient temperature for 24 hours. The mixture was then filtered and the purple/black solution concentrated to give purple/black crystals of the intended product (0.37 g, 99%). LCMS (Method A) (RT 2.53 minutes [M+H]⁺ 324.; ¹H NMR (400 MHz, CDCl₃) δ H 1.40 (s, 9H), 6.50 (s, 1H), 6.82 (m, 3H), 7.28 (m, 2H), 7.53 (d, J=7.62 Hz, 1H), 8.16 (d, J=8.23 Hz, 1H).

### Reference Example 10

### Synthesis of 2-[4-(1-Benzyloxycarbonyl-piperidin-4-ylcarbamoyl)-1H-pyrazol-3-yl]-1H-benzoimidazole-5-carboxylic Acid

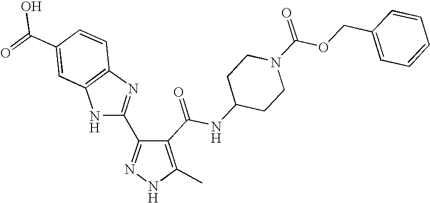

2-[5-Methyl-4-(piperidin-4-ylcarbamoyl)-1H-pyrazol-3-yl]-3H-benzimidazole-5-carboxylic acid ethyl ester (586 mg, 1.10 mmol) was added to a 1:1 mix of 2N NaOH and methanol (20 mL). The mixture was heated to 60° C. After one hour, the methanol was removed under reduced pressure and the aqueous solution was neutralised by addition of 10% HCl. The solution was extracted with ethyl acetate and washed with water. After drying the solution was concentrated to give a white powder. (500 mg, 90%). LCMS (RT 2.17 minutes, [M+H]⁺ 503); ¹H NMR (400 MHz, CD₃OD) δ H 1.75 (m, 2H), 3.13 (m, 2H), 2.65 (s, 3H), 3.42 (m, 2H), 4.11 (m, 2H), 4.22 (m, 1H), 5.20 (s, 2H), 7.40 (m, 7H), 7.61 (d, J=8.47 Hz, 1H), 7.99 (broad, 1H), 8.31 (br, 1H).

### Reference Example 11

### Synthesis of 1-Chloro-2-isopropenyl-4-nitrobenzene

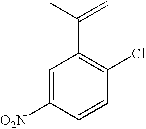

A solution of methyltriphenylphosphonium bromide (1.07 g, 3.0 mmol) in THF (10 ml) was added n-butyllithium (2.5M hexane solution, 1.21 ml, 3.0 mmol) 0° C. under nitrogen. After the mixture was stirred for 5 min, 2-chloro-5-nitroacetophenone (0.5 g, 2.5 mmol) in THF (3 ml) was added drop-wise at 0° C. The mixture was maintained at 0° C. then allowed to warm to ambient temperature overnight. The reaction mixture was cooled and quenched with 1N HCl (aq) (20 ml). The solution was extracted with ethyl acetate and washed with brine, dried (MgSO₄) and condensed. The resultant yellow oil residue was subjected to flash column chromatography on silica eluting hexane to give title compound (0.17 g, 35%) as a clear oil; ¹H-NMR (400 MHz, CDCl₃) δ 2.13 (1H, s broad), 5.07 (1H, s broad), 5.36 (1H, s broad), 7.53 (1H d), 8.08 (2H, m aromatic).

### Reference Example 12

### Synthesis of 4-Chloro-3-isopropyl-phenylamine

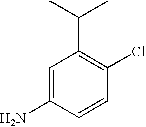

A mixture of 1-chloro-2-isopropenyl-4-nitrobenzene (0.134 g, 0.68 mmol) and 5% Pt/C (0.020 g, catalytic) in ethanol (5 ml) was agitated under an atmosphere of hydrogen at room temperature for 2 hr. The reaction mixture was filtered to remove catalyst and evaporated to give the title compound as brown oil (0.114 g, 99%); LC/MS: RT 2.38 (Method A) [M+H]⁺ 170; ¹H-NMR (400 MHz, CDCl₃) δ 1.20 (6H, d), 3.31 (1H, m), 6.51 (1H, dd), 6.66 (1H, d), 7.10 (1H, d).

### Reference Example 13

### Synthesis of 4-Chloro-5-isopropyl-2-nitro-phenylamine

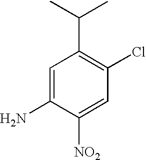

A solution of 4-chloro-3-isopropyl-phenylamine (2.1 μg, 12.48 mmol) in trifluoroacetic anhydride (20 ml) was treated, in portion, with KNO₃ (1.32 g, 13.06 mmol) at 0° C. The solution was stirred in ice bath for 2 hr and then allowed to warm to ambient temperature over 1 hr. The solution was diluted with ice-water (100 ml) and then extracted twice with dichloromethane. The combined organics were washed with brine, dried (MgSO₄) and condensed. The resultant orange oil was dissolved in methanol (30 ml) and treated with 7% (w/w) aqueous K₂CO₃ (20 ml) at room temperature. The reaction mixture was allowed to stir overnight at room temperature. The methanol was removed under pressure, diluted with water (50 ml) and extracted three times with dichloromethane. The combined organics were washed with brine, dried (MgSO₄) and condensed. The resultant brown solid residue was subjected to flash column chromatography on silica eluting gradient of hexane to (1:9, v/v) ethyl acetate and hexane to give title compound (1.92 g, 72%) as a orange solid; ¹H-NMR (400 MHz, CDCl₃) δ 1.80 (6H, d), 3.87 (1H, m), 7.82 (1H, s), 8.69 (1H, s).

### Reference Example 14

### Synthesis of 3-(5-Chloro-6-isopropyl-1H-benzoimidazol-2-yl)-5-methyl-1H-pyrazole-4-carboxylic Acid Ethyl Ester

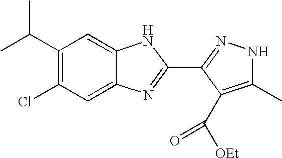

A suspension of 4-chloro-5-isopropyl-2-nitro-phenylamine (1 g, 4.66 mmol) and 3-formyl-5-methyl-1H-pyrazole-4-carboxylic acid ethyl ester (prepared by methods analogous to Reference Examples 5 and 6, 0.85 g, 4.66 mmol) was treated with 1M (aq) Na₂S₂O₄ (2.43 g, 13.97 mmol) and heated at 90° C. overnight. The cooled reaction mixture was treated drop-wise with 5N (aq) NH₄OH (30 ml). A precipitate was immediately formed which was then filtered, washed with water and dried under vacuum to afford title compound (1.01 g, 63%) as white solid; LC/MS: RT 2.50 (Method A) [M+H]⁺ 347; ¹H-NMR (400 MHz, D6 DMSO+TFA) δ 1.25 (9H, m), 2.53 (3H, s), 3.41 (1H, m), 4.32 (2H, q), 7.84 (1H, s broad), 7.92 (1H, s broad).

### Reference Example 15

### 4-Cyclopropyl-2-nitro-phenylamine

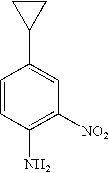

4-Cyclopropyl-2-nitro-phenylamine was prepared starting from 4-bromo-2-nitroaniline by methods analogous to reference Example 9; LC/MS: RT 2.30 (Method A) [M+H]⁺ 179.

### Reference Example 16

### 3-(6-Cyclopropyl-1H-benzoimidazol-2-yl)-5-methyl-1H-pyrazole-4-carboxylic Acid Ethyl Ester

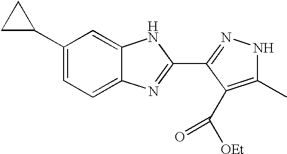

3-(6-Cyclopropyl-1H-benzoimidazol-2-yl)-5-methyl-1H-pyrazole-4-carboxylic acid ethyl ester was prepared by methods analogous to reference Example 14; LC/MS: RT 1.95 (Method A) [M+H]⁺ 311.

### Example 1

### Synthesis of 3-(1H-Benzoimidazol-2-yl)-5-methyl-1H-pyrazole-4-carboxylic Acid piperidin-4-ylamide

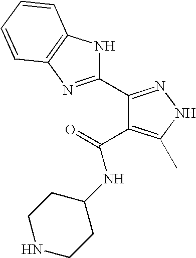

A mixture of 3-tert-butoxymethyl-5-methyl-1H-pyrazole-4-carboxylic acid (500 mg, 2.36 mmol), EDC (1.35 g, 3 eq), HOBt (975 mg, 3 eq), 4-amino-(1N-tertbutoxycarbamate)piperidine (945 mg, 2 eq) and DIPEA (1.25 mL, 3 eq) in DMF (40 mL) was heated at 50° C. for 4.5 h, then allowed to cool. The mixture was evaporated and partitioned between chloroform and water. The layers were separated, and the aqueous phase was extracted with chloroform (×2). The combined organic extracts were washed with brine, dried (MgSO₄) and condensed. The crude product was crystallised/triturated with hot EtOAc-hexane (1:4; 20 mL), cooled, filtered, washed with EtOAc-hexane, and sucked dry to afford 3-hydroxymethyl-5-methyl-1H-pyrazole-4-carboxylic acid piperidin-4-ylamide (545 mg, 58%) as a pale orange powder.

To a stirred solution of 3-tert-butoxymethyl-5-methyl-1H-pyrazole-4-carboxylic acid piperidin-4-ylamide (200 mg, 0.51 mmol) in DCM (3 mL), cooled in ice-water, was added trifluoroacetic acid (3 mL). The stirred reaction was allowed to warm to ambient temperature and was stirred for 4.5 h. The mixture was evaporated to afford crude 3-hydroxymethyl-5-methyl-1H-pyrazole-4-carboxylic acid piperidin-4-ylamide (395 mg) which was used in the next step without further purification

3-Hydroxymethyl-5-methyl-1H-pyrazole-4-carboxylic acid piperidin-4-ylamide (assumed 0.51 mmol) was treated with manganese dioxide (85%; 500 mg, 10 eq) in DME (10 mL). After 1 h stirring at ambient temperature, the mixture was heated at reflux for 45 min. After this time, MeOH was added and the resultant hot suspension was filtered through celite and eluted with hot MeOH. Evaporation afforded 3-formyl-5-methyl-1H-pyrazole-4-carboxylic acid piperidin-4-ylamide as an amber oil which was used directly in the next step.

To a solution of 3-formyl-5-methyl-1H-pyrazole-4-carboxylic acid piperidin-4-ylamide (assume 0.51 mmol) in DMF (3 mL) was added EtOH (1 mL), 1,2-phenylenediamine (55 mg, 1 eq) and sodium bisulfite (95 mg, 1.8 eq). The mixture was heated in a sealed microwave vessel at 160° C. for 10 min. Evaporation and purification by reverse phase preparative HPLC (Method B) afforded the title compound as a pale yellow foam/solid (67.4 mg). R^{e}— purification of 22 mg of this material (reverse phase preparative HPLC, method B) afforded an analytically pure sample of 3-(1H-Benzoimidazol-2-yl)-5-methyl-1H-pyrazole-4-carboxylic acid piperidin-4-ylamide as an off-white solid (14 mg).

LC/MS retention time 1.38 minutes (Method A) m/z 325 (M+H, 100%). 1H-NMR (400 MHz; d⁶-DMSO) δ H 11.98 (1H, d, J=6.6 Hz), 8.42 (1H, s), 7.63 (2H, br s), 7.24-7.30 (2H, m), 4.02-4.11 (2H, m), 3.20-3.28 (2H, m), 2.92-3.01 (2H, m), 2.57 (3H, s), 2.00-2.08 (2H, m) and 1.70-1.82 (2H, m).

### Example 2

### Synthesis of 3-(5,6-Dimethyl-1H-benzoimidazol-2-yl)-5-methyl-1H-pyrazole-4-carboxylic Acid piperidin-3-ylamide

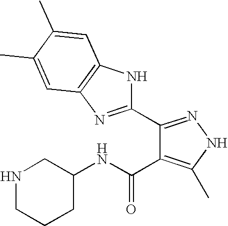

A mixture of 3-(5,6-Dimethyl-1H-benzoimidazol-2-yl)-5-methyl-1H-pyrazole-4-carboxylic acid (Prepared in analogous manner to Ref Example 7) (0.99 g) EDC (0.85 g), HOBt (0.54 g), 3-amino-piperidine-1-carboxylic acid benzyl ester (0.94 g) and TEA (1.1 mls) in DMF (100 mls) was stirred at 50° C. for 16 hrs. The mixture was diluted in ethyl acetate (200 mls) and washed with 1N HCl (aq) (50 mls), 5% sodium bicarbonate (aq) (50 mls) & brine (50 mls). The organics were dried with anhydrous magnesium sulphate, filtered and then passed through a pad of silica eluting neat ethyl acetate. Selected fractions were combined and evaporated, the residue brought to a solid by trituration in diethyl ether and solid collected by filtration and dried. The solid was dissolved in DMF (200 mls) and hydrogenated at atmospheric pressure over 10% palladium on carbon at 60° C. for 48 hrs. The reaction mixture was filtered to remove catalyst and evaporated. The residue was again brought to a solid by trituration with methanol and collection by filtration gave title compound (0.78 g) (LC/MS RT: 1.73, [M+H]⁺ 352), ¹H-NMR (400 MHz, D6 DMSO) δ H 1.44-1.47 (4H m), 2.73 (2H s broad), 2.94 (2H s broad), 2.33 (6H s), 2.57 (3H s), 2.63 (2H t), 2.80 (2H d), 3.04 (2H d), 3.83 (2H, m), 11.82 (2H d)

### Example 3

### Synthesis of 3-(5,6-Dimethyl-1H benzoimidazole-2-yl)-5-methyl-1H-pyrazole-4-carboxylic Acid (1-methyl-piperidin-3-yl)-amide

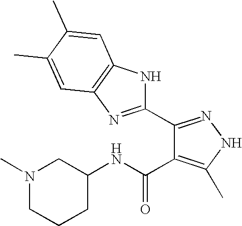

(0.1 g) was dissolved in methanol (100 mls) and treated with excess aqueous formaldehyde solution (33% by volume) (1 ml) and hydrogenated for 16 hrs. The catalyst was removed by filtration and the residue purified by flash column chromatography eluting a mixture of 10% methanol, 2% 7N ammonia in methanol, the remainder dichloromethane. Selected fractions were evaporated and the residue solid was triturated with acetonitrile and collected by filtration. The colourless solid was washed further with diethyl ether and dried under vacuum to afford title compound 0.062 g. (LC/MS RT: 1.76, [M+H]⁺ 367) (¹H-NMR-D6 DMSO) δ H 1.45-1.56 (2H m), 1.83 (2H s broad), 2.20 (3H s), 2.33 (6H s), 2.56 (3H s), 3.97 (1H broad), 7.28 (1H s aromatic), 7.40 (1H s aromatic)

### Example 4

### Synthesis of 3-(1H-Benzoimidazol-2-yl)-5-methyl-1H-pyrazole-4-carboxylic Acid (3-amino-propyl)-amide

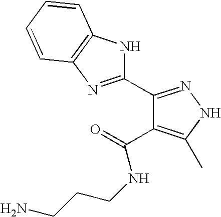

(3-{[3-(1H-Benzoimidazol-2-yl)-5-methyl-1H-pyrazole-4-carbonyl]-amino}-propyl)-carbamic acid tert-butyl ester (0.04 g) was stirred in dichloromethane (5 mls) and treated with trifluoroacetic acid (5 mls) and the mixture stirred under nitrogen for 2 hrs. The reaction mixture was evaporated to dryness and re-evaporated twice from toluene. The residue was triturated with diethyl ether and title compound obtained by filtration (0.04 g 97%) (LC/MS RT: 1.45, [M+H]⁺ 298), ¹H-NMR (400 MHz D6 DMSO) δ H 1.91 (2H m), 2.57 (3H s), 2.98 (2H s broad), 3.45 (2H m), 7.30 (2H m), 7.65 (1H s broad), 7.78 (2H s broad)

### Example 5

### Synthesis of 5-Methyl-3-(1H-naphtho[2,3-d]imidazol-2-yl)-1H-pyrazole-4-carboxylic Acid piperidine-4-ylamide

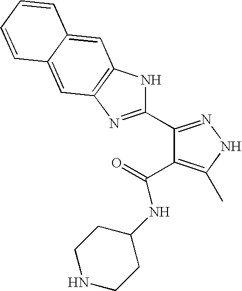

A mixture of 3-tert-Butoxymethyl-5-methyl-1H-pyrazole-4-carboxylic acid (3.71 g), EDC (4 g), HOBt (2.84 g), 4-amino(1N-tertbutoxycarbamate) piperidine (3.5 g) and TEA (7.2 mls) in DMF was stirred to 50° C. for 16 hrs. The mixture was diluted in ethyl acetate (200 mls) and washed with 1N HCl (aq) (50 mls), 5% sodium bicarbonate (aq) (50 mls) & brine (50 mls). The organics were dried with anhydrous magnesium sulphate, filtered and evaporated. The residue was treated with trifluoroacetic acid (10 mls) and stirred for 3 hrs. The mixture was re-evaporated and dried through azeotrope with toluene. The resultant 3-hydroxymethyl-5-methyl-1H-pyrazole-4-carboxylic acid piperidin-4-ylamide was treated with di-tert-butyl dicarbonate (2.68 g) and TEA (3.4 mls) in dichloromethane at room temperature under nitrogen. The reaction mixture was diluted with ethyl acetate washed with 1N HCl (aq), 5% sodium bicarbonate (aq), and the organic layer dried over anhydrous magnesium sulphate and filtered. The filtrate was evaporated and treated with manganese dioxide (0.87 g) in DME (20 mls) and heated to 80° C. for 2 hrs. The mixture was filtered hot through celite, the manganese further washed with warm methanol. The combined filtrate was evaporated and the residue in acetonitrile (50 mls) was treated with 2,3-diaminonapthylene (0.32 g) and sodium bisulphite (0.42 g) and the mixture refluxed for 16 hrs. The reaction mixture was cooled, reduced in volume and partitioned between ethyl acetate and 1N HCl (aq), and the organic layer washed with 5% sodium bicarbonate (aq) and brine. The organics were again dried and evaporated. The residue purified by flash column chromatography eluting neat ethyl acetate (RF 0.30). Fractions were collected and evaporated to residue solid. The residue was dissolved in methanol and treated with excess HCl dioxane and stirred under nitrogen at room temperature for 3 hrs. The reaction mixture was evaporated to dryness and the residue flashed eluting dichloromethane containing 10% (2N ammonia in methanol). Fractions were collected and evaporated, the resultant colourless residue was triturated with acetonitrile and filtration gave the title compound (0.31 g 4%) (LC/MS: RT 1.78 [M+H]⁺ 375) ¹H-NMR (400 MHz D6 DMSO) δ H 1.89 (2H m), 2.10 (2H d), 2.60 (3H s), 3.05 (2H t), 3.31 (2H m), 4.13 (1H m), 7.41 (2H d), 8.04 (2H d), 12.03 (1H d)

### Example 6

### Synthesis of 2-[5-Methyl-4-(piperidin-4-ylcarbamoyl)-1H-pyrazol-3-yl]-3H-benzimidazole-5-carboxylic Acid Ethyl Ester

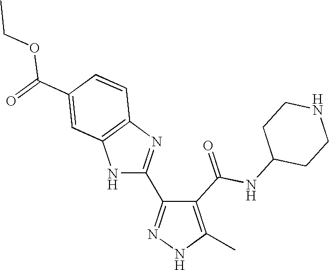

4-[(3-Formyl-1H-pyrazole-4-carbonyl)-amino]-piperidine-1-carboxylic acid benzyl ester (50 mg, 0.135 mmol) was slurried in acetonitrile (20 mL). 3,4-Diamino-benzoic acid ethyl ester (25 mg, 0.135 mmol) and sodium bisulphite (17 mg, 0.162 mmol) were added. The mixture was heated to reflux for 48 hours, after which time it was concentrated, taken up in ethyl acetate and washed with water, then brine. The organics were then dried, concentrated and taken up in DMF (20 mL). A catalytic amount of palladium on carbon was added and the mixture was degassed and hydrogenated at 60° C. for 24 hours. The mixture was then filtered, concentrated and purified by reverse-phase HPLC (Method B) to give the title compound as a cream coloured solid (10.1 mg, 15%); LCMS (RT 1.72 minutes, [M+H]⁺ 397); ¹H NMR (400 MHz, CD₃OD) δ H 1.32 (m, 3H), 1.65 (m, 2H), 1.95 (m, 2H), 2.22 (m, 2H), 2.54 (s, 3H), 3.17 (m, 2H), 3.84 (m, 1H), 4.32 (m, 2H), 7.66 (d, J=8.53 Hz, 1H), 7.88 (d, J=8.49 Hz, 1H), 8.21 (s, 1H).

### Example 7

### Synthesis of 2-[5-Methyl-4-(piperidin-4-ylcarbamoyl)-1H-pyrazol-3-yl]-3H-benzimidazole-5-carboxylic Acid Cyclopentylamide

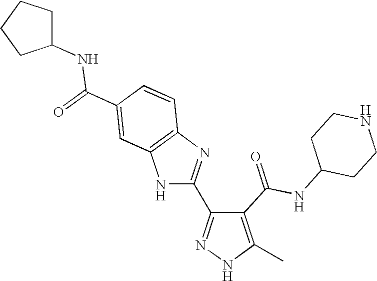

2-[4-(Piperidin-4-ylcarbamoyl)-1H-pyrazol-3-yl]-1H-benzoimidazole-5-carboxylic acid (50 mg, 0.1 mmol) was added to DMF (10 mL). To this mixture was added EDCI (57 mg, 0.299 mmol), HOBt (40 mg, 0.299 mmol), and Diisopropylethylamine (0.05 mL, 0.299 mmol). Cyclopentylamine (25 mg, 0.299 mmol) was added and the mixture was stirred at room temperature for two hours. The mixture was partitioned between ethyl acetate and water; the resultant organic layer was washed once with water, 1N HCl and brine. After drying, the solution was concentrated to give a yellow oil which was taken up in DMF (20 mL). A catalytic amount of palladium on carbon was added and the mixture was degassed and hydrogenated at 60° C. for 24 hours. The mixture was then filtered, concentrated and purified by reverse-phase HPLC (Method B) to give the title compound as a cream coloured solid (8.67 mg, 20%). LCMS 1.684 MIN 436.10; ¹H NMR (400 MHz, CD₃OD) δ H 1.55 (m, 4H), 1.70 (m, 4H), 1.97 (m, 4H), 2.53 (s, 3H), 2.76 (m, 4H), 3.99 (m, 1H), 4.27 (m, 1H), 7.55 (d, J=8.45 Hz, 1H), 7.67 (d, J=8.44 Hz, 1H), 8.03 (s, 1H).

### Example 8

### Synthesis of 3-[6-(1H-Indol-2-yl)-1H-benzimidazo-2-yl]-5-methyl-1H-pyrazole-4-carboxylic Acid piperidin-4-ylamide

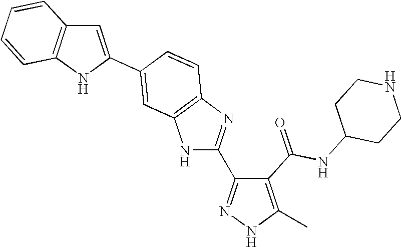

4-[(3-Formyl-1H-pyrazole-4-carbonyl)-amino]-piperidine-1-carboxylic acid benzyl ester (50 mg, 0.135 mmol) was slurried in acetonitrile (10 mL). To this was added 2-(3,4-Diamino-phenyl)-indole-1-carboxylic acid tert-butyl ester (44 mg, 0.135 mmol). Sodium bisulphite (23 mg, 0.27 mmol) was added and the mixture was heated to reflux. After 24 hours the mixture was concentrated and partitioned between ethyl acetate and water. After washing with brine, the organics were dried and concentrated to give an orange oil. Dichloromethane (1 mL) was added, followed by TFA (5 mL) and the mixture stirred at room temperature for 2 hours. The mixture was then concentrated by azeotroping twice with toluene to give a dark green oil. DMF was added, followed by a catalytic amount of Palladium on carbon and the mixture degassed and hydrogenated at 60° C. After 24 hours the mixture was filtered, concentrated and purified by preparative scale HPLC to give a cream coloured solid (5.9 mg, 10%). LCMS (RT 1.94 minutes, [M+H]⁺ 440); ¹H NMR (400 MHz, CD₃OD) δ H 1.88 (m, 2H), 2.18 (m, 2H), 2.70 (s, 3H), 3.15 (m, 2H), 3.47 (m, 2H), 4.12 (m, 1H), 6.89 (t, J=7.40 Hz, 1H), 7.01 (t, J=7.38 Hz, 1H), 7.28 (d, J=8.05 Hz, 1H), 7.36 (d, J=8.58 Hz, 1H), 7.63 (d, J=9.26 Hz, 1H), 7.67 (s, 1H), 7.98 (s, 1H).

Examples 9 to 47 in the following tables were prepared by methods analogous to Examples 1 to 8 above. All 36 compounds were tested for activity in kinase assays described below in the Assay section. The result obtained in each case is given.

All examples demonstrate unexpected selectivity for PDK1 and CHK1 versus similar serine/threonine kinases Akt1 (>70 fold), PKA (>30 fold) & CDK2 (>15 fold). Broad kinase screening performed at Upstate showed that examples (1 & 5) demonstrate >20 fold selectivity verses Abl(h), CK1e(h), CK2(h), IKKa(h), JNK1a1(h), MEK1(h), MKK6(h), NEK2(h), PDGFRa(h), PLk3(h), ROCK-II(h), SAPK2a(h). Further, example (14) showed >20 fold selectivity versus EGFR, FGFR1, FGFR2, FGFR3, FGFR4, Flt1 IGF-IR, ITK, KDR, PDGFRa, Syk, Tie2.

| | | | PDK-1 | CHK-1 | AKT-1 | PKA | CDK-2 |
|---|---|---|---|---|---|---|---|
| Example | | LC/MS | IC50 | IC50 | IC50 | IC50 | IC50 |
| Number | Structure | Method A | (μM) | (μM) | (μM) | (μM) | (μM) |
| 1 | 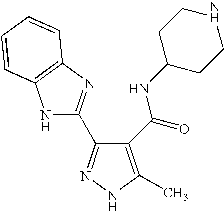 | RT 1.38 mins Mass ion [M + H]⁺ 325 | 0.108 | 0.098 | 17.07 | 7.26 | 3.30 |
| 2 | 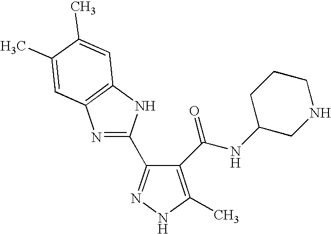 | RT 1.73 mins Mass ion [M + H]⁺ 353 | 0.219 | 0.055 | 15.90 | 7.84 | 3.60 |
| 3 | 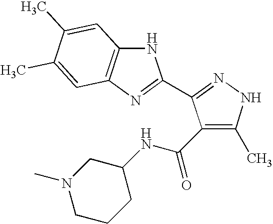 | RT 1.76 mins Mass ion [M + H]⁺ 367 | 0.440 | 0.077 | 41.33 | 23.40 | 30.90 |
| 4 | 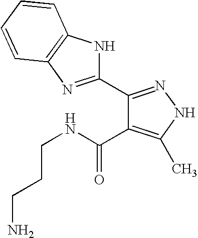 | RT 1.45 mins Mass ion [M + H]⁺ 299 | 0.212 | 0.031 | >50 | 20.71 | 39.78 |
| 5 | 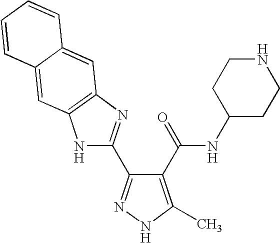 | RT 1.78 mins Mass ion [M + H]⁺ 375 | 0.058 | 0.010 | >50 | 3.24 | 4.54 |
| 6 | 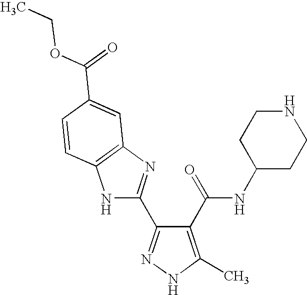 | RT 1.72 mins Mass ion [M + H]⁺ 397 | 0.433 | 0.023 | >50 | 40.18 | 39.47 |
| 7 | 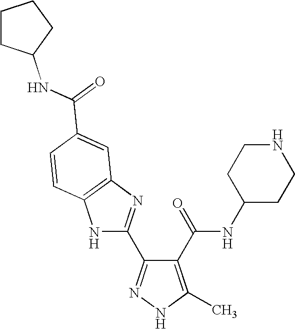 | RT 1.68 mins Mass ion [M + H]⁺ 436 | 0.070 | 0.043 | 28.60 | 12.58 | 9.24 |
| 8 | 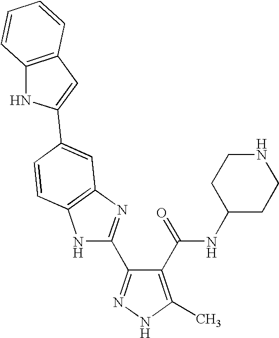 | RT 1.94 mins Mass ion [M + H]⁺ 440 | 0.019 | 0.012 | 5.84 | 1.34 | 2.80 |
| 9 | 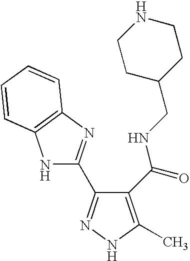 | RT 1.54 mins Mass ion [M + H]⁺ 339 | 0.129 | 0.208 | 11.13 | 7.51 | 47.04 |
| 10 | 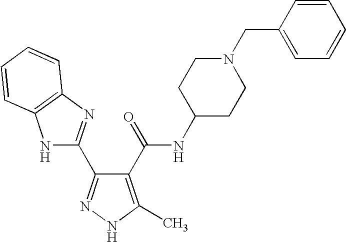 | RT 1.81 mins Mass ion [M + H]⁺ 415 | 0.435 | 0.107 | >50 | 29.14 | 43.97 |
| 11 | 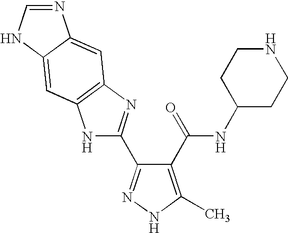 | RT 1.30 mins Mass ion [M + H]⁺ 365 | 0.120 | 0.088 | >50 | 7.77 | 38.93 |
| 12 | 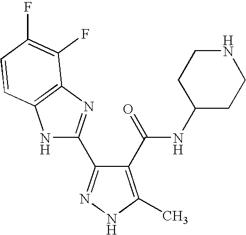 | RT 1.63 mins Mass ion [M + H]⁺ 361 | 0.485 | 0.609 | >50 | 20.88 | 10.51 |
| 13 | 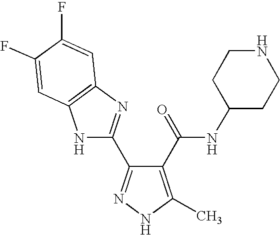 | RT 1.61 mins Mass ion [M + H]⁺ 361 | 0.520 | 0.508 | >50 | 19.31 | 7.92 |
| 14 | 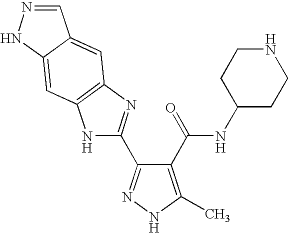 | RT 1.38 mins Mass ion [M + H]⁺ 365 | 0.010 | 0.006 | 26.84 | 7.20 | 5.11 |
| 15 | 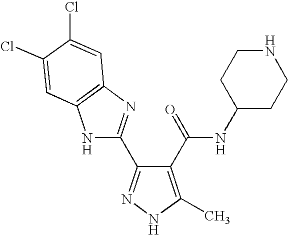 | RT 1.76 mins Mass ion [M + H]⁺ 393/395 | 0.123 | 0.295 | >50 | 8.85 | 4.77 |
| 16 | 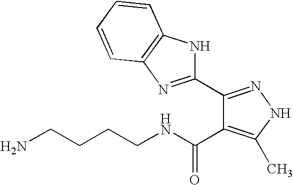 | RT 1.52 mins Mass ion [M + H]⁺ 313 | 0.249 | 0.213 | >50 | 20.69 | 43.51 |
| 17 | 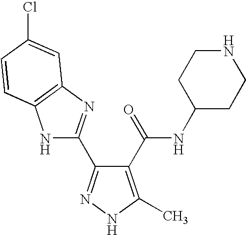 | RT 1.66 mins Mass ion [M + H]⁺ 359 | 0.189 | 0.222 | >50 | 9.03 | 10.61 |
| 18 | 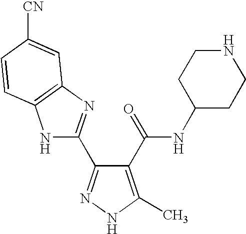 | RT 1.54 mins Mass ion [M + H]⁺ 350 | 0.311 | 0.137 | >50 | 22.58 | 12.19 |
| 19 | 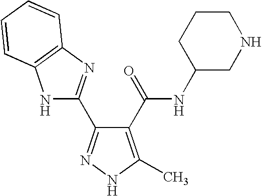 | RT 1.58 mins Mass ion [M + H]⁺ 325 | 0.050 | 0.058 | >50 | 10.89 | 4.47 |
| 20 | 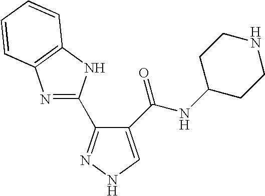 | RT 1.46 mins Mass ion [M + H]⁺ 311 | 0.323 | 0.252 | >50 | 32.09 | 6.36 |
| 21 | 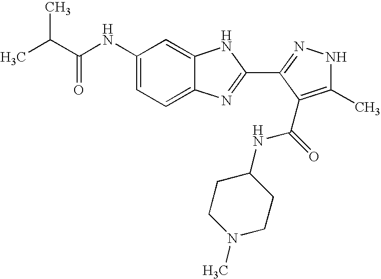 | RT 1.54 mins Mass ion [M + H]⁺ 424 | 0.074 | 0.006 | >50 | 13.10 | 21.30 |
| 22 | 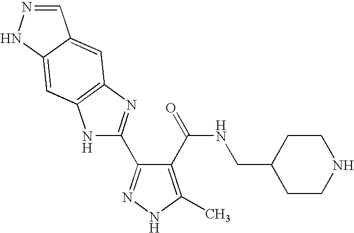 | RT 1.83 mins Mass ion [M + H]⁺ 379 | 0.110 | 0.094 | 36.13 | 6.40 | 31.05 |
| 23 | 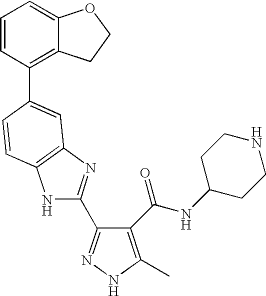 | RT 1.85 mins Mass ion [M + H]⁺ 443 | 0.125 | 0.015 | 25.57 | 5.36 | 21.10 |
| 24 | 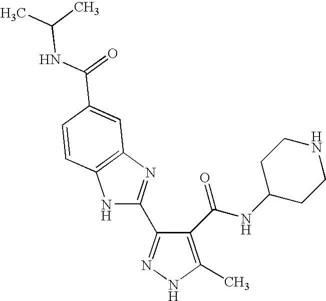 | RT 1.57 mins Mass ion [M + H]⁺ 410 | 0.046 | 0.036 | 34.46 | 10.71 | 6.42 |
| 25 | 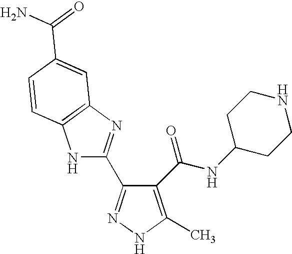 | RT 1.49 mins Mass ion [M + H]⁺ 396 | 0.084 | 0.068 | >50 | 13.02 | 5.10 |
| 26 | 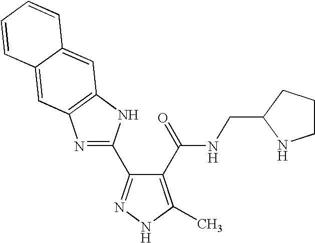 | RT 1.88 mins Mass ion [M + H]⁺ 375 | 0.521 | 0.309 | >50 | 34.69 | 34.25 |
| 27 | 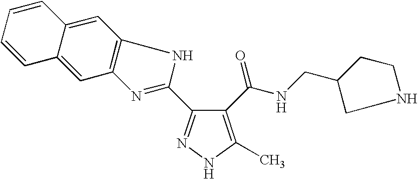 | RT 1.77 mins Mass ion [M + H]⁺ 375 | 0.143 | 0.004 | >50 | 8.31 | 6.24 |
| 28 | 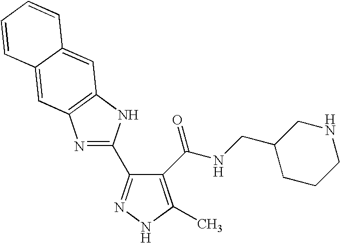 | RT 1.84 mins Mass ion [M + H]⁺ 389 | 0.125 | 0.008 | >50 | 8.99 | 8.50 |
| 29 | 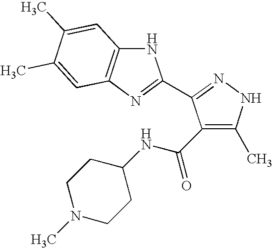 | RT 1.58 mins Mass ion [M + H]⁺ 367 | 0.180 | 0.016 | >50 | 5.98 | 8.24 |
| 30 | 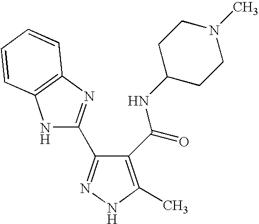 | RT 1.50 mins Mass ion [M + H]⁺ 339 | 0.139 | 0.0635 | 11.75 | 20.247 | 2.213 |
| 31 | 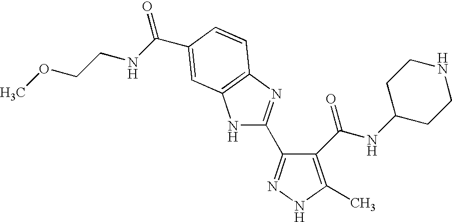 | RT 1.45 mins Mass ion [M + H]⁺ 426 | 0.073 | 0.002 | 31.37 | 8.04 | 11.42 |
| 32 | 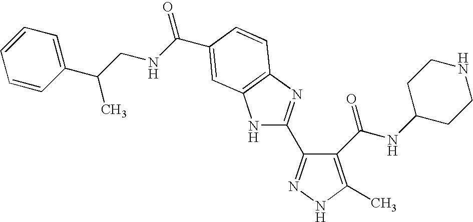 | RT 1.79 mins Mass ion [M + H]⁺ 486 | 0.036 | 0.003 | 18.14 | 6.50 | 15.60 |
| 33 | 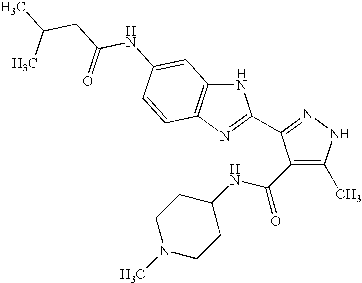 | RT 1.59 mins Mass ion [M + H]⁺ 438 | 0.095 | 0.003 | 47.51 | 13.10 | 21.30 |
| 34 | 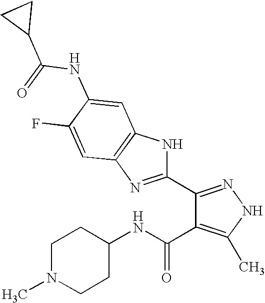 | RT 1.52 mins Mass ion [M + H]⁺ 440 | 0.216 | 0.013 | 44.11 | 28.40 | 20.6 |
| 35 | 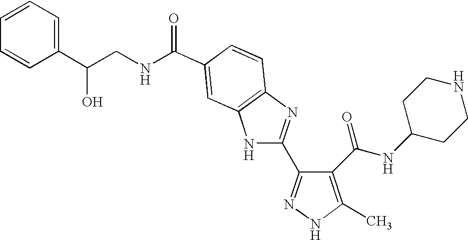 | RT 1.58 mins Mass ion [M + H]⁺ 488 | 0.057 | 0.037 | 10.648 | 5.314 | 13.783 |
| 36 | 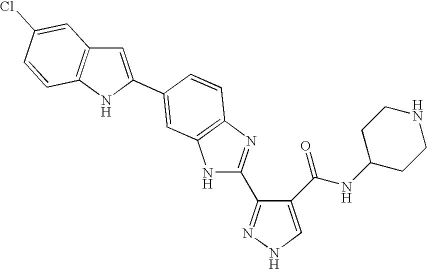 | RT 1.95 mins Mass ion [M + H]⁺ 474 | 0.155 | 0.058 | >10 | 3.620 | 12.772 |
| 37 | 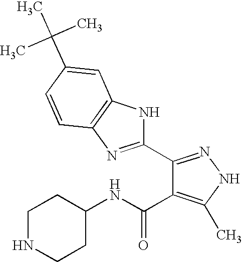 | RT 1.59 mins Mass ion [M + H]⁺ 381 | 0.049 | 0.006 | >10 | 4.949 | 1.854 |
| 38 | 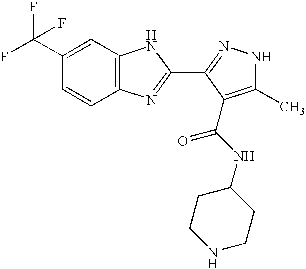 | RT 1.54 mins Mass ion [M + H]⁺ 393 | 0.032 | 0.058 | >10 | 16.869 | 19.307 |
| 39 | 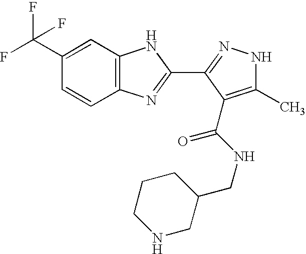 | RT 1.63 mins Mass ion [M + H]⁺ 407 | 0.086 | 0.049 | >10 | >50 | >50 |
| 40 | 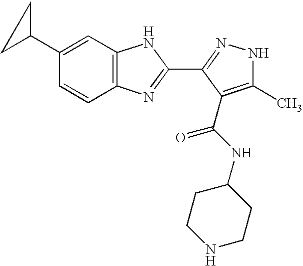 | RT 1.56 mins Mass ion [M + H]⁺ 365 | 0.020 | 0.022 | >10 | 2.856 | 23.706 |
| 41 | 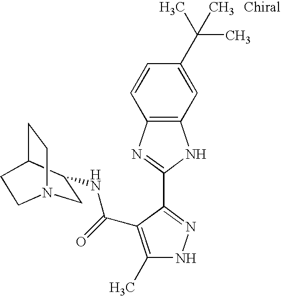 | RT 1.77 mins Mass ion [M + H]⁺ 407 | 0.052 | 0.025 | >10 | 2.124 | >50 |
| 42 | 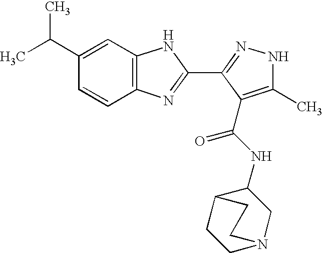 | RT 1.74 mins Mass ion [M + H]⁺ 393 | 0.045 | 0.050 | >10 | 3.820 | 9.675 |
| 43 | 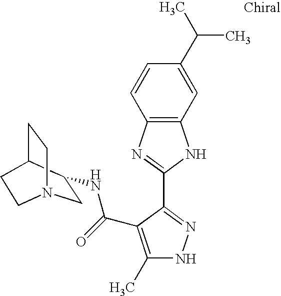 | RT 1.74 mins Mass ion [M + H]⁺ 393 | 0.022 | 0.026 | >10 | 2.018 | 14.689 |
| 44 | 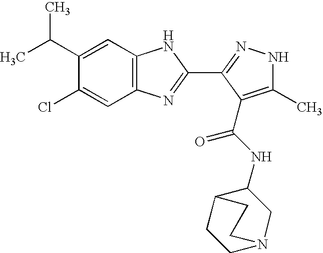 | RT 1.89 mins Mass ion [M + H]⁺ 427 | 0.140 | 0.091 | >10 | 12.023 | >50 |
| 45 | 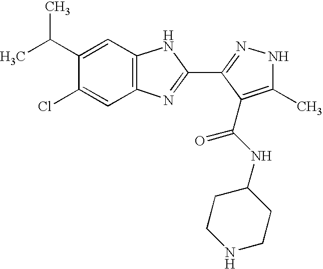 | RT 1.76 mins Mass ion [M + H]⁺ 401 | 0.013 | 0.013 | >10 | 5.128 | 11.894 |
| 46 | 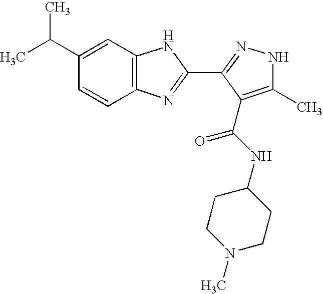 | RT 1.63 mins Mass ion [M + H]⁺ 381 | 0.036 | 0.014 | >10 | 3.805 | 24.162 |
| 47 | 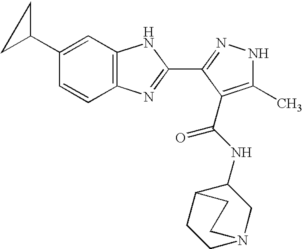 | RT 1.68 mins Mass ion [M + H]⁺ 391 | 0.106 | 0.085 | >10 | 3.825 | >50 |

The following example has an IC50<0.05 μM vs. PDK-1

| Example | | LCMS |
|---|---|---|
| Number | Structure | Method A |
| 48 | 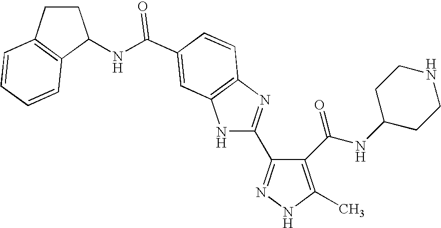 | RT 1.77 mins Mass ion [M + H]⁺ 484 |

The following examples have an IC50<0.5 μM vs. PDK-1 & CHK-1

| Example | | LCMS |
|---|---|---|
| Number | Structure | Method A |
| 49 | 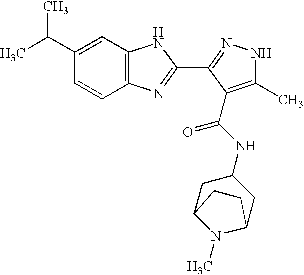 | RT 1.72 mins Mass ion [M + H]⁺ 407 |
| 50 | 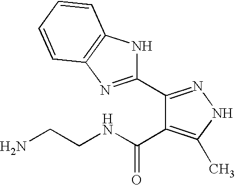 | RT 1.46 mins Mass ion [M + H]⁺ 285 |

In the examples, characterization and/or purification were performed using standard spectroscopic and chromatographic techniques, including liquid chromatography-mass spectroscopy (LC-MS) and high performance liquid chromatography (HPLC), using the conditions described in methods A and B. NMR experiments were conducted on a Bruker DPX400 ultra shield NMR spectrometer in the specified solvent. Reactions carried out under microwave irradiation were conducted in a Smith Synthesizer.

### LCMS Method A

Instrument: HP1100
Column: Luna 3 μm, C18(2), 30 mm×4.6 mm i.d. from Phenomenex
Temperature: 22° C.
Solvents: A—Water+10 mmol/L ammonium acetate+0.08% (v/v) formic acid
   B—95% Acetonitrile-5% Solvent A+0.08% (v/v) formic acid
Gradient:

| | | | Flow |
|---|---|---|---|
| Time (min) | Solvent A (%) | Solvent B (%) | (cm³min⁻¹) |
| 0 | 95 | 5 | 2 |
| 0.25 | 95 | 5 | 2 |
| 2.50 | 5 | 95 | 2 |
| 2.55 | 5 | 95 | 3 |
| 3.60 | 5 | 95 | 3 |
| 3.65 | 5 | 95 | 2 |
| 3.70 | 5 | 95 | 2 |
| 3.75 | 95 | 5 | 2 |

Detection: UV detection at 230, 254 and 270 nm
Mass Spec: HP1100 MSD, series A
   Ionization was positive or negative ion electrospray
   Molecular weight scan range was 120-1000

### Method B

Instrument: Waters FractionLynx MS autopurification system
Column: Luna 5 μm, C18(2), 100 mm×21.2 mm i.d. from Phenomenex
Temp: ambient
Solvents: A—water+0.08% (v/v) formic acid
   B—95% methanol-water+0.08% (v/v) formic acid
Flow rate: 20 cm³ min⁻¹
Gradient:

| Time (min) | Solvent A (%) | Solvent B (%) |
|---|---|---|
| 0 | 95 | 5 |
| 0.5 | 50 | 50 |
| 7.0 | 20 | 80 |
| 7.5 | 5 | 95 |
| 9.5 | 5 | 95 |
| 10.0 | 95 | 5 |

Detection: Photodiode array 210 to 400 nm
Mass spec: MicroMass ZQ
   Ionization was positive or negative ion electrospray
   Molecular weight scan range was 150-1000
Collection: Triggered on selected mass ion

### Assay Protocols

### PDK1

Assays for the PDK dependent kinase activity were carried out by monitoring the phosphorylation of a synthetic peptide, KTFCGTPEYLAPEVRREPRILSEEEQEMFRDFDYIADWC. The assay mixture containing the inhibitor and PDK1 enzyme was mixed together in a microtiter plate in a final volume of 50 μl and incubated for 60 min at 30° C. The assay mixture contained 0.01 mM unlabeled ATP, 0.01 μCi/μl ³³P-γ-ATP, 0.075 mM peptide, 0.1 mg/ml BSA, 7.5 mM magnesium acetate, 0.05M Tris.HCl, pH 7.5, 0.5% 2-mercaptoethanol. The reaction was stopped by adding 50 μl of 50 mM phosphoric acid. 90 μl of the mixture were transferred to a pre-wetted 96-well Multiscreen MAPHNOB filtration plate (Millipore) and filtered on a vacuum manifold. The filter plate was washed with 3 successive additions of 200 μl 50 mM phosphoric acid and then with 100 μl methanol. The filtration plate was dried for 10 min at 65° C., scintillant added and phosphorylated peptide quantified in a scintillation counter (Trilux, PerkinElmer)

### CHK1

Assays for the Chk1 kinase activity were carried out by monitoring the phosphorylation of a synthetic peptide Chktide with the amino acid sequence, KKKVSRSGLYRSPSMPENLNRPR. The assay mixture containing the inhibitor and Chk1 enzyme was mixed together in a microtiter plate in a final volume of 50 μl and incubated for 40 minutes at 30° C. The assay mixture contained 0.01 mM unlabeled ATP, 0.5 Ci ³³P-γ-ATP, 30 μM Chktide, 0.1 mg/ml BSA, 50 mM Hepes-NaOH pH 7.5 and 11 nM GST-Chk1 enzyme. The reaction was stopped by adding 50 μl of 50 mM phosphoric acid. 90 μl of the mixture was transferred to a pre-wetted 96-well multi-screen MAPHNOB filtration plate (Millipore) and filtered on a vacuum manifold. The filter plate was washed with 3 successive additions of 200 μl 50 mM phosphoric acid and then with 100 μl methanol. The filtration plate was dried for 10 min at 65° C., scintillant added and phosphorylated peptide quantified in a scintillation counter (Trilux, PerkinElmer)

### CDK2

Assays for the cyclin dependent kinase activity were carried out by monitoring the phosphorylation of a synthetic peptide, HATTPKKKRK. The assay mixture containing the inhibitor and CDK-2 enzyme, complexed with cyclin A (0.4 U/ml) was mixed together in a microtiter plate in a final volume of 50 μl and incubated for 40 min at 30° C. The assay mixture contained 0.1 mM unlabeled ATP, 0.01 μCi/μl ³³P-γ-ATP, 0.03 mM peptide, 0.1 mg/ml BSA, 7.5 mM magnesium acetate, 50 mM HEPES-NaOH, pH 7.5. The reaction was stopped by adding 50 μl of 50 mM phosphoric acid. 90 μl of the mixture were transferred to a pre-wetted 96-well Multiscreen MAPHNOB filtration plate (Millipore) and filtered on a vacuum manifold. The filter plate was washed with 3 successive additions of 200 μl 50 mM phosphoric acid and then with 100 μl methanol. The filtration plate was dried for 10 min at 65° C., scintillant added and phosphorylated peptide quantified in a scintillation counter (Trilux, PerkinElmer)

HEPES is N-[2-Hydroxyethyl]piperazine-N′-[2-ethanesulfonic acid] BSA is bovine serum albumin.

### Akt1

Protocol for the Akt assay is the same for CDK2 except that, 3 nM Akt1 (Upstate) was used with Histone H1 as substrate with a final ATP concentration of 200 μM. The reaction was incubated at 30° C. for 40 minutes.

### PKA

Protocol for the PKA assay is the same for CDK2 except that, 10 units of PKA enzyme (Upstate) were used with 0.75 μM Kemptide as a substrate with a final ATP concentration of 100 μM. The PKA assay was incubated at 30° C. for 30 minutes.

1. A compound of formula (I) or a salt, hydrate or solvate thereof 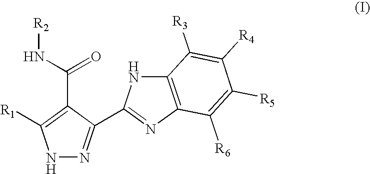
wherein
R₁ is hydrogen or C₁-C₃ alkyl;
R₂ is a radical of formula R₇(CH₂)ₙ—, or a radical of formula -Alk-N(—R₈)—R₉ wherein n is 0, 1, 2 or 3 and Alk is C₁-C₆ alkylene;
R₃ and R₆ are independently selected from hydrogen, fluoro, or chloro;
R₄ and R₅ are independently selected from hydrogen, C₁-C₆ alkyl, halo, cyano, C₁-C₆ alkoxycarbonyl, C₁-C₆ alkoxy, trifluoromethyl, —C(═O)—NH—R₁₀, NH—C(═O)—R₁₁, a heterocyclic ring optionally substituted by halo, or a C₃-C₆ cycloalkyl ring;
or R₄ and R₅ taken together with the carbon atoms to which they are attached form a 5- or 6-membered carbocyclic ring, or a 5- or 6-membered heterocyclic ring;
R₇ is (i) a heterocyclic ring of 5 or 6 ring atoms coupled via a ring carbon wherein the sole heteroatom is nitrogen, optionally substituted by C₁-C₆ alkyl or aryl C₁-C₆ alkyl, (ii) 1-aza-bicyclo[2.2.2]oct-3-yl, or (iii) 8-methyl-8-azabicyclo[3.2.1]oct-3-yl;
R₈ and R₉ are independently selected from hydrogen or C₁-C₃ alkyl;
R₁₀ and R₁₁ are independently selected from hydrogen, C₃-C₇ cycloalkyl, C₁-C₆ alkyl, C1-C₆ alkoxy-C₁-C₆ alkyl, aryl, or aryl-C₁-C₆ alkyl wherein the C₁-C₆ alkyl part is optionally substituted by hydroxy.

2. A compound as claimed in claim 1 wherein R₁ is hydrogen.

3. A compound as claimed in claim 1 wherein R₁ is methyl.

4. A compound as claimed in claim 1 wherein R₂ is piperidin-4-yl, piperidin-3-yl, piperidin-4-ylmethyl, piperidin-3-ylmethyl, 1benzyl-piperidin-4-yl, 4-amino-butyl, 3-amino-propyl, pyrrolidin-2-ylmethyl, pyrrolidin-3-ylmethyl, 1-methyl-piperidin-4-yl, 1-methyl-piperidin-3-yl, 2-aminoethyl, or 1-aza-bicyclo[2.2.2]oct-3-yl.

5. A compound as claimed in claim 1 wherein R₂ is piperidin-4-yl.

6. A compound as claimed in claim 1 wherein R₃ is fluoro.

7. A compound as claimed in claim 1 wherein R₃ is hydrogen.

8. A compound as claimed in claim 1 wherein R₆ is fluoro.

9. A compound as claimed in claim 1 wherein R₆ is hydrogen.

10. A compound as claimed in claim 1 wherein R₄ is a heterocyclic ring containing at least one donor nitrogen atom.

11. A compound as claimed in claim 1 wherein R₅ is a heterocyclic ring containing at least one donor nitrogen atom.

12. A compound as claimed in claim 1 wherein R₄ and R₅ are independently selected from hydrogen, methyl, fluoro, chloro, cyano, ethoxycarbonyl, aminocarbonyl, isopropylaminocarbonyl, cyclopentylaminocarbonyl, 2-methoxyethylaminocarbonyl, 2,3-dihydroindan-1-ylaminocarbonyl, 2-phenylpropylaminocarbonyl, isopropylcarbonylamino, isobutylcarbonylamino, cyclopropylcarbonylamino, cyclopentylcarbonylamino, indol-2-yl, and 2,3-dihydrobenzofuran-4-yl.

13. A compound as claimed in claim 1 wherein and R₄ and R₅ taken together with the carbon atoms to which they are attached form a 5- or 6-membered heterocyclic ring containing at least one donor nitrogen atom.

14. A compound as claimed in claim 1 wherein R₄ and R₅ taken together with the carbon atoms to which they are attached form a benzene ring, a 4,5-fused imidazole ring, or a 4,5-fused pyrazole ring.

15. A compound of formula (II) or a salt, hydrate or solvate thereof 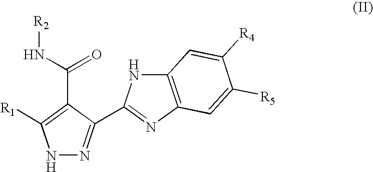
wherein
R₁ is hydrogen or methyl;
R₂ is piperidin-4-yl, pyrrolidin-3-ylmethyl, 1-methyl-piperidin-4-yl, or 1-aza-bicyclo[2.2.2]oct-3-yl;
R₄ and R₅ are independently selected from hydrogen, C₁-C₆ alkyl, —C(═O)—NH—R₁₀, a heterocyclic ring optionally substituted by halo, or a C₃-C₆ cycloalkyl ring;
or R₄ and R₅ taken together with the carbon atoms to which they are attached form a 5- or 6-membered carbocyclic ring, or a 5- or 6-membered heterocyclic ring containing at least one donor nitrogen atom;
R₁₀ is C₁-C₆ alkyl, cyclopropyl, 2-methoxyethyl, 2-phenylpropyl, or 2-phenylethyl.

16. A compound as claimed in claim 15 wherein R₄ and R₅ taken together with the carbon atoms to which they are attached form a benzene ring, or a 4,5-fused pyrazole ring.

17. A compound as claimed in claim 15 wherein R₄ and R₅ are independently selected from hydrogen, isopropyl, cyclopropyl, tert-butyl, or 1H-indol-2-yl.

18. A compound as claimed in claim 15 wherein R10 is isopropyl or isobutyl.

19. A pharmaceutical composition comprising a compound as claimed in claim 1 and a pharmaceutically acceptable carrier.

20. (canceled)

21. A method of treatment of a mammal suffering from a condition responsive to inhibition of PDK1 and CHK1 activity, comprising administering to the mammal an amount of a compound as claimed in claim 1 effective to inhibit PDK1 and CHK1 activity in the mammal.

22. The method as claimed in claim 21 wherein the condition responsive to inhibition of PDK1 and CHK1 activity is selected from cancer and autoimmune disorders.

23. A method as claimed in claim 22 wherein said autoimmune disorder is organ transplant rejection, lupus, multiple sclerosis, rheumatoid arthritis and osteoarthritis.

24. A method as claimed in claim 22 for cancer by selective inhibition of PDK1 and CHK1 activity over PKA and/or CDK-2 and/or AKT-1 activity.
